(19) United States
(12) Patent Application Publication
(10) Pub. No.: US 2019/0139012 A1
(43) Pub. Date: May 9, 2019

(54) System and method for billing healthcare services

(71) Applicant: Prairie Spine and Pain Institute, Peoria, IL (US)

(72) Inventors: Richard A. Kube, II, Peoria, IL (US); Colleen Ingraham, Peoria, IL (US)

(21) Appl. No.: 15/998,629

(22) Filed: Aug. 16, 2018

Related U.S. Application Data
(60) Provisional application No. 62/546,415, filed on Aug. 16, 2017.

(51) Int. Cl. G06Q 20/14; G06F 16/23; G16H 10/60; G06Q 40/08

(52) U.S. Cl. CPC G06Q 20/14; G06Q 40/08; G16H 10/60; G06F 16/2379

(57) ABSTRACT

The present disclosure provides a system. In an embodiment, a system for billing healthcare services is provided and includes one or more computing devices. The one or more computing devices include a memory module, an interface, and a processing module. The processing module includes a billing module and an integration module. The memory module stores (i) patient historical data, (ii) a universal charge master database, (iii) a medical billing code database, (iv) a payor fee schedule database, and (v) an entire health encounter (EHE) database. The EHE database includes a unique identifier for each EHE. For each EHE, the unique identifier includes a custom code, a procedure name, a description of EHE services, a list of EHE-related medical billing codes, and a contract rate for the EHE. Receipt of the custom code by the billing module triggers the integration module to perform the following functions: (vi) calculate a universal charge rate for the EHE based on the EHE-related medical billing codes and the universal charge master; (vii) attach the custom code to the contract rate for a specified payor; (viii) apply a discount for the difference between the universal charge rate and the contract rate; and (ix) generate a bill for healthcare service for the specified payor. The bill has a 1-line entry with the custom code, the procedure name, and the contract rate.

### BACKGROUND

In most markets, prices and quality indicators are transparent—clear and readily available to consumers. Health care is different. For healthcare, prices are difficult to obtain and are often meaningless when they are disclosed. Patients who ask for price information from a healthcare service provider are likely to receive little or no information. Typically, neither a hospital nor a doctor will know the cost until the procedure is completed. It is generally recognized that the U.S. healthcare system makes it difficult to find information on quality and cost of care. This hidden information is putting patients at risk. This secrecy puts everyone from consumers to corporations at an unfair disadvantage—leading to gaps in quality of care and much higher costs.

The healthcare industry historically has used a variety of medical codes to document and bill for healthcare services and supplies. Recently, a variety of payors and providers in the healthcare industry have proposed a bundled payment model, or “global fee model,” in an attempt to improve price transparency. The global fee model combines all fees for healthcare services into a single fee. For example, services provided by surgeons, surgical assists, anesthesiologists, certified registered nurse anesthetists, radiology/imaging, pathology, implants, disposables, hospitals and facilities are to be combined into a single fee under the global fee model.

The global fee model presently does not exist outside of the hospital setting. For non-hospital healthcare providers, conventional medical billing systems either (1) use a generic and pre-determined written description of the medical procedure or (2) expand the meaning of an existing medical billing code to generate an invoice.

These current medical billing systems have deficiencies. In scenario (1), no way presently exists for the medical billing system to integrate the description of the medical procedure into the healthcare provider's practice management system. Consequently, it is inefficient and time consuming (i) to create an invoice and/or, (ii) to manage and track payments and adjustments, etc., based upon generic boilerplate written descriptions of medical procedures that are pre-installed into conventional medical billing systems. This scenario (1) would require tracking to occur outside of the existing practice management systems.

Current medical record/billing systems rely upon the condition that one and only one meaning is applicable for a given individual medical billing code. To expand the meaning for a code to mean something more than its original definition creates problems. In scenario (2), expanding the definition of existing medical billing codes creates confusion and likelihood for human error. For example, no rule or formula exists in current healthcare practice management systems to determine when to use a given CPT definition. A user (a healthcare provider or a payor, for example) of an incumbent medical record/billing system must choose, on a case-by-case basis whether to use the traditional definition of an AMA CPT code or whether to use an expanded definition for a given CPT code. This introduces uncertainty into the medical billing cycle because healthcare employees not trained in medical billing are called to make billing/charge decisions beyond their skill sets—leading to confusion and ambiguity. Scenario (2) also eliminates the healthcare provider's ability to have a universal charge master for each medical billing code. Since a given medical billing code can often mean different things in Scenario (2), the same medical billing code oftentimes carries different fees under different situations, causing even more confusion. The uncertainty under Scenario (2) exposes a healthcare provider to the risk of being accused as having a fluid charge master, which could be construed as a violation of standard medical services payor contracts, such as contracts with Medicare and Medicaid.

A need exists for a healthcare billing methodology that converts the descriptions of bundled services into a code that readily translates into language used in existing practice management software. A need further exists for healthcare billing methodology that gives each medical billing code a distinct and constant definition allowing for a universal charge master, and for revenue cycle management staff to implement the same into their existing medical billing systems with minimal training and virtually no change in workflows.

### SUMMARY

The present disclosure provides a system. In an embodiment, a system for billing healthcare services is provided and includes one or more computing devices. The one or more computing devices include a memory module, an interface, and a processing module. The processing module includes a billing module and an integration module. The memory module stores (i) patient historical data, (ii) a universal charge master database, (iii) a medical billing code database, (iv) a payor fee schedule database, and (v) an entire health encounter (EHE) database. The EHE database includes a unique identifier for each EHE. For each EHE, the unique identifier includes a custom code, a procedure name, a description of EHE services, a list of EHE-related medical billing codes, and a contract rate for the EHE. Receipt of the custom code by the billing module triggers the integration module to perform the following functions: (vi) calculate a universal charge rate for the EHE based on the EHE-related medical billing codes and the universal charge master; (vii) attach the custom code to the contract rate for a specified payor; (viii) apply a discount for the difference between the universal charge rate and the contract rate; and (ix) generate a bill for healthcare services for the specified payor. The bill has a 1-line entry with the custom code, the procedure name, and the contract rate.

The present disclosure provides a method. In an embodiment, a method for billing healthcare services is provided and includes preparing a list of medical procedures necessary for providing an entire healthcare encounter (EHE) to a patient. The method includes linking the EHE with two or more relevant medical billing codes. The method includes determining a contract rate for the EHE. The method includes creating a unique identifier for the EHE. The unique identifier includes (i) a custom code, (ii) a procedure name, (iii) a description of EHE services, (iv) a list of EHE-related medical billing codes, and (v) the contract rate for the EHE. The method includes calculating a universal charge for the EHE from a universal charge master and from the EHE-related CMS codes. The method includes attaching the custom code to the contract rate for a specified payor. The method includes applying a discount for the difference between the universal charge rate and the contract rate. The method includes generating a bill for healthcare services for the specified payor. The bill has a 1-line entry with the custom code, the procedure name, and the contract rate.

### BRIEF DESCRIPTION OF THE DRAWINGS

FIG. 1 is a block diagram of a system for billing healthcare services in accordance with an embodiment of the present disclosure.

FIG. 2 is a table of unique identifiers for respective entire healthcare encounters in accordance with an embodiment of the present disclosure.

FIG. 3 is a flow diagram illustration of the techniques performed by an integration module in accordance with embodiments of the present disclosure.

FIG. 4 is a prior art bill for healthcare services with multiple line charge entries.

FIG. 5 is a bill for healthcare services generated by the present system and a 1-line entry in accordance with the present disclosure.

### DEFINITIONS

For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

The numerical ranges disclosed herein include all values from, and including, the lower and upper value. For ranges containing explicit values (e.g., 1 or 2, or 3 to 5, or 6, or 7), any subrange between any two explicit values is included (e.g., 1 to 2; 2 to 6; 5 to 7; 3 to 7; 5 to 6; etc.).

Unless stated to the contrary, implicit from the context, or customary in the art, all parts and percents are based on weight and all test methods are current as of the filing date of this disclosure.

The term “communicates” or “in communication with,” or “communicatively connected,” or “communicatively linked” and like terms denotes a link between two or more objects (i.e., a link between two or more modules, units, sub-units, computing devices, processors, servers, etc.) that enables two-way exchange of information and includes a wired connection, a wireless connection, and combinations thereof. The term “communicatively connected” is a link between two or more objects (i.e., a link between two or more modules, units, sub-units, computing devices, servers) that enables two-way exchange of information and includes a wired connection, a wireless connection, and combinations thereof.

The terms “comprising,” “including,” “having,” and their derivatives, are not intended to exclude the presence of any additional element, component, step or procedure, whether or not the same is specifically disclosed. In contrast, the term, “consisting essentially of” excludes from the scope of any succeeding recitation any other element, component, step or procedure, excepting those that are not essential to operability. The term “consisting of” excludes any element, component, step, or procedure not specifically delineated or listed.

A “computing device” (or “a computer readable device”) is a non-transitory computing device with a central processing unit (CPU), random access memory (RAM), and a storage medium (such as hard disk drive, solid state drive, flash memory, cloud storage). Nonlimiting examples of computing devices include personal computers (PCs), smart phones, laptops, mobile computing devices, tablet PCs, and servers. The term “computing device” may also describe two or more computing devices communicatively linked in a manner as to distribute and share one or more resources, such as clustered computing devices and server banks/farms. It is understood that any number of computing devices could be used, and embodiments of the present disclosure are contemplated for use with any computing device.

A “healthcare provider,” as used herein, is a provider of medical services or health services and any other person or organization who furnishes, medical bills, or is paid for health care in the normal course of business. Nonlimiting examples of healthcare providers include healthcare providers/entities that are recognized and/or defined under the United States Code of Federal Regulations, as a doctor of medicine or osteopathy, podiatrist, dentist, chiropractor, clinical psychologist, optometrist, nurse practitioner, nurse-midwife, or a clinical social worker who is authorized to practice by a State of the United States and performing within the scope of their practice as defined by State law.

The “Internet” reters to interconnected (public and/or private) networks that may be linked together by protocols (such as TCP/IP and HTTP) to form a globally accessible distributed network. While the term Internet refers to what is currently known (e.g., a publicly accessible distributed network), it also encompasses variations which may be made in the future, including new protocols or any changes or additions to existing protocols.

A “medical billing code,” as used herein, is an alpha-numeric identifier for a product and/or service provided in the healthcare industry. Nonlimiting examples of medical billing codes include the International Classification of Diseases (ICD) codes (including versions 9 and 10), Current Procedural Technology (CPT) codes (maintained by the American Medical Association or AMA), Healthcare Common Procedural Coding System codes (HCPCS), Physician Quality Reporting System (PQRS) codes, Diagnosis Related Group (DRG) codes, and any combination thereof.

A “server” is a computer program that provides services to other computer programs (and their users) in the same or other computing devices. The computing device that a server program runs in is also frequently referred to as a server (though it may be used for other purposes as well). In the client/server programming model, a server is a program that awaits and fulfills requests from client programs in the same or other computing devices. A given application in a computing device may function as a client with requests for services from other programs and also as a server of requests from other programs. Specific to the Web, a Web server is the computer program (housed in a computing device) that serves requested HTML pages or files. A Web client is the requesting program associated with the user. For example, the Web browser in a home PC is a client that requests HTML files from a Web server.

“Wireless communication” is one or more wireless technologies such as Near Field Communications (NFC), Wi-Fi, infrared, Bluetooth, or one or more variants of wireless cellular technology.

A “web site” refers to a system that serves content over a network using the protocols of the World Wide Web. A web site may correspond to an Internet domain name, and may serve content associated or provided by an organization. The term may encompass (i) the hardware/software server components that serve objects and/or content over a network, and/or (ii) the “backend” hardware/software components, including any standard, non-standard or specialized components, that may interact with the server components that provide services for Web site users.

The “World Wide Web” (or “Web”) refers to (i) a distributed collection of user-viewable or accessible documents (that may be referred to as Web documents or Web pages) or objects that may be accessible via a publicly accessible distributed network like the Internet, and/or (ii) the client and server software components which provide user access to documents and objects using communication protocols. A protocol that may be used to locate, deliver, or acquire Web documents or objects through HTTP (or other protocols), and the Web pages may be encoded using HTML, tags, and/or scripts. The terms “Web” and “World Wide Web” encompass other languages and transport protocols including or in addition to HTML and HTTP that may include security features, server-side, and/or client-side scripting.

### DETAILED DESCRIPTION

The present disclosure is directed to a system and a method for billing healthcare services.

### 1. System

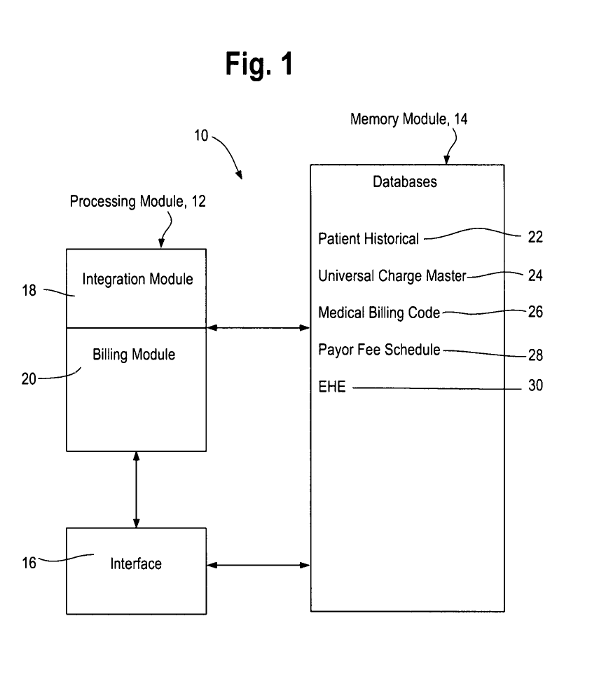

In an embodiment, the present system is accomplished through the use of one or more computing devices. FIG. 1 shows a system 10 for billing healthcare services. System 10 includes a processing module 12, a memory module 14, and a user interface 16. The processing module 16 includes an integration module 18, and a billing module 20.

The processing module 12 may include a general purpose microprocessor, a specifically-designed processor, an application specific integrated circuit, a field programmable gate array, a collection of discrete logic, any combination of the foregoing devices, or any type of processing device capable of executing the techniques described herein. The techniques of this disclosure may be implemented at least partially in hardware, such as a processor or discrete logic circuits. The techniques may also be implemented using aspects of software or firmware in combination with the hardware. If implemented at least partially in software or firmware, the software or firmware may be executed in one or more hardware processors, such as a microprocessor, application specific integrated circuit (ASIC), field programmable gate array (FPGA), or digital signal processor (DSP). The software that executes the techniques may be initially stored in a computer-readable storage medium and loaded and executed in the processor. The processor may execute modules (i.e., the integration module, and/or the billing module) to perform the techniques of this disclosure, and the modules may comprise combinations of software and hardware, e.g., software routines executing on the processor.

The memory module 14 includes one or more databases for storing information in the system 10. A user may enter information into the system 10 by way of the interface 16. The interface 16 includes a graphical user interface (a display screen and a keyboard), a printing device, and may optionally include connectivity to the Internet. In other words, the interface 16 enables a user to communicate with the processing module 12 and/or the memory module 14, and vice versa. Communication between the components of the system 10 may be by way of hard wire communication, wireless communication, communication via the Internet, or communication via the World Wide Web.

In an embodiment, one or more databases in the memory module 14 store program instructions (e.g., software instructions) that are executed by the processing module 12 to carry out, or otherwise to perform, the techniques described herein.

The memory module 14 includes one or more databases for storing information. Nonlimiting examples of information stored in the memory module 14 includes patient demographics (for example age, gender, predisposition to diseases, among others), patient medical insurance eligibility (such as copay, coinsurance, deductibles among others), existing medical insurance coverage, patient reimbursement history, and any combination thereof. This patient history is hereafter referred to as “patient historical data” or patient historical database 22.

The memory module 14 also includes one or more databases for storing universal chargemaster data 24. Universal chargemaster data may include thousands of healthcare services, medical procedures, equipment fees, drugs, supplies, and diagnostic evaluations. Each item in the chargemaster is assigned a unique medical billing code and a set charge or a set fee. In the universal chargemaster database, a charge (or a fee) is attached to each medical billing code based upon researched data for the usual and customary price for a medical billing code in a healthcare provider's geographic area. The assignment of a price to a medical billing code is what becomes the universal chargemaster—that is, the list of all billable medical codes and the set price for each medical billing code. The set price for each medical billing code is uniformly applied, or otherwise universally applied, to all payors—consequently, the designation of the term “universal chargemaster database.”

The memory module 14 also includes one or more databases for storing medical billing code data 26. The medical billing code data may include codes for thousands of healthcare services, medical procedures, equipment fees, drugs, supplies, and diagnostic evaluations. The medical billing code database 26 may or may not be a sub-database of the universal chargemaster database 24.

The memory module 14 includes one or more databases for storing payor fee schedule data 28. The payor fee schedule database 28 includes data and information related to fee arrangements between one or more specified payors and a healthcare provider. A “payor fee schedule,” as used herein, is a pre-negotiated and contracted fee arrangement for healthcare services provided by a healthcare provider and to be paid by a payor. A payor fee schedule is not an arbitrary arrangement; rather the payor fee schedule is a pre-approved and negotiated contract, mutually agreed-upon by both the healthcare provider and the specified payor for fees to be paid by the payor for healthcare services provided by, or to be provided by, the healthcare provider. A “payor,” as used herein, is an entity or an individual responsible for paying the bill for healthcare services. Nonlimiting examples of payors include a governmental entity (Medicare, Medicaid), an insurance company, a third party administrator, an employer (such as a self-insured employer, for example,) and an individual (such as a patient, for example), and any combination thereof.

The memory module 14 of the system 10 includes an entire healthcare encounter (EHE) database 30 for storing entire healthcare encounter data. An “entire healthcare encounter, or “EHE” is a list of medical procedures necessary for providing an entire healthcare encounter to a patient. The “entire healthcare encounter” is the aggregate of health care services, healthcare facilities, medical equipment, medical disposables, and miscellaneous medical resources necessary to provide a pre-determined healthcare procedure to a patient. In other words, the list of medical procedures necessary to provide the EHE includes everything, or substantially everything, necessary to administer the EHE to the patient. Nonlimiting examples of items to be included in the EHE include healthcare providers (surgeon, surgical assistant, anesthesiologist, nurse, certified registered nurse anesthetist, radiologist, medical technician (imaging technician, for example), medical laboratory testing (pathologist), hospital facilities (operation room, recovery room, intensive care unit), surgical equipment, implant devices, medical disposables (needles, intravenous bags/tubing), and any combination thereof.

More than one healthcare provider is typically required to administer the EHE to the patient. In an embodiment, a lead healthcare provider (1HP) prepares the EHE (such as a surgical practice group, for example). The lead healthcare provider (1HP) is an entity with one or more employees which may include healthcare providers/professionals (doctors, nurses), medical billing experts, administrative personnel, and any combination thereof. One or more employees of the 1HP devise the list of medical procedures necessary to provide the EHE. The employees of the 1HP also identify all auxiliary healthcare providers (in this example, radiologist, anesthesiologist, pathologist to support the surgeon in an EHE that is a surgical procedure), medical subcontractors, medical supply vendors, and healthcare facilities (hospital, operating room, recovery room) necessary to administer the EHE, hereafter collectively referred to as secondary healthcare providers, or “2HP.” In an embodiment, the 1HP contracts in advance with each 2HP for the price/cost of each 2HP's contribution to the EHE.

In an embodiment, the 1HP creates a unique identifier for the EHE. The unique identifier includes a custom code, a procedure name, a description of EHE services (EHE descriptor), a list of EHE-related medical billing codes, and a contract rate for each respective EHE. A user (such as an employee of the 1HP, for example) enters the unique identifier by way of the interface 16 and processing module 12 for storage in the EHE database 30 of the memory module 14.

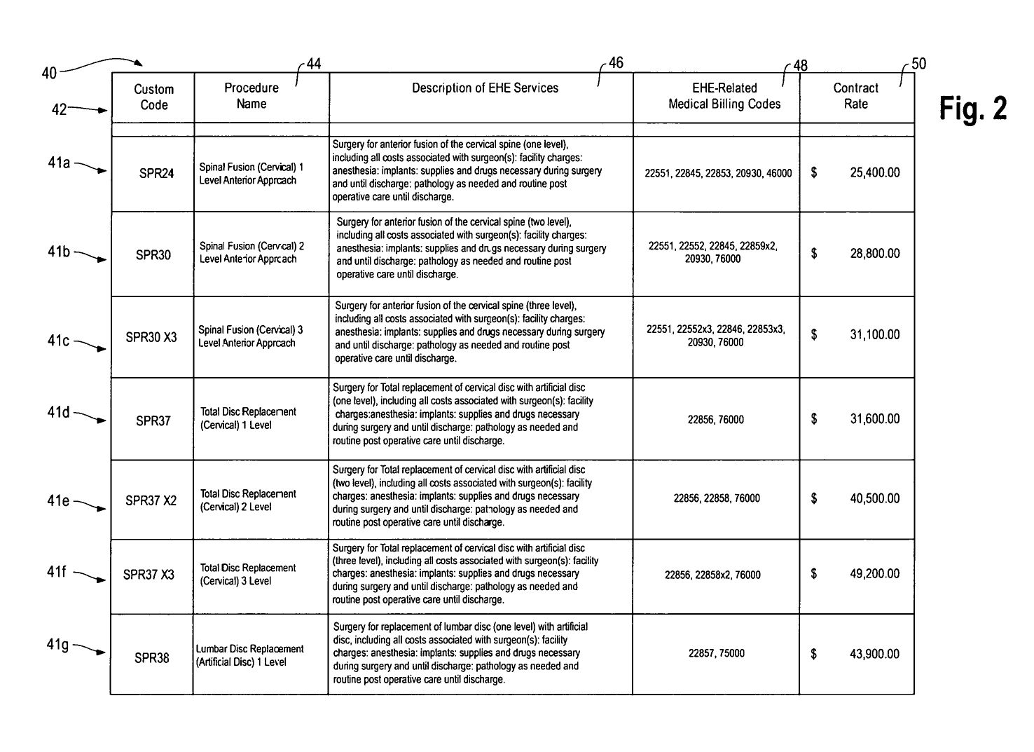

FIG. 2 shows a table 40 with seven nonlimiting examples of unique identifiers 41a-41g for seven respective EHEs. Each unique identifier 41a-41g includes a custom code 42, a procedure name 44, a description of EHE services (EHE descriptor) 46, a list of EHE-related medical billing codes 48, and a contract rate 50. Although FIG. 2 shows seven unique identifiers 41a-41g, it is understood that any number (less than seven or more than seven) of unique identifiers and EHEs may be stored in the memory module 14.

In an embodiment, the 1HP determines the medical billing codes that are related to the administration of the EHE for the creation of the EHE-related medical billing codes 48.

In an embodiment, the 1HP determines the contract rate 50 for the EHE. The 1HP determines the contract rate based on (i) the 1HP's costs associated for providing its portion of the EHE and (ii) the costs associated with the 2HP contracts for providing the EHE. In a further embodiment, one or more payors agree to (i.e., “contract to”) the contract rate 50 with 1HP in advance of the EHE being administered to a patient covered by the payor. In other words, the contract rate 50 is a fee/price that is mutually-agreed upon between a specified payor and the 1HP prior to the EHE being administered to a patient for whom the payor is responsible for payment.

The unique identifier—and the custom code 42 in particular—is not CPT code or an ICD code. In an embodiment, the unique identifier is created by the 1HP and is entered into the system 10 by way of the interface 16 and the processing module 12. A user can also print a copy of the unique identifier (or any portion thereof) using the interface 16. FIG. 2 shows a nonlimiting example of Table 40 with seven unique identifiers retrieved from the memory module 14 and printed via interface 16.

A user (such as an employee of the 1HP, for example) through the interface 16, selects a billing mode in system 10. User selection of the billing mode activates, or otherwise launches, the billing module 22. When the user desires to bill a payor for an EHE (typically after administration of the EHE to the patient), the user, in billing mode, inputs the custom code (custom code 42) into the interface 16. In the billing module 20, the user inputs the custom code 42 (i.e., “SPR24” from Table 40 of FIG. 2) into the interface 16. The integration module 18 recognizes the custom code that is input into the billing module 20, which triggers activation of the integration module 18.

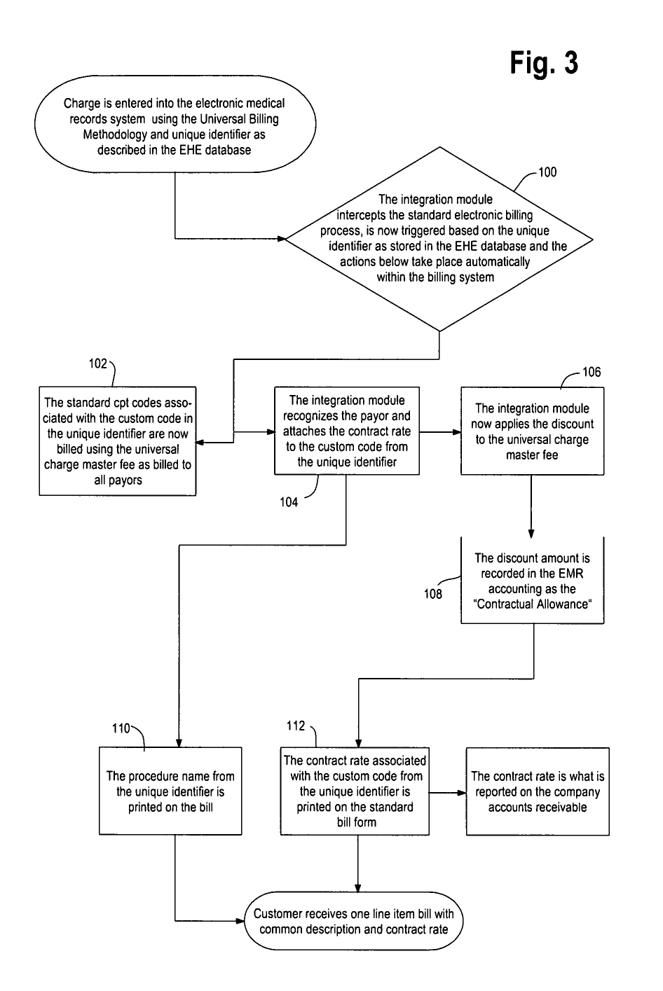

FIG. 3 shows the flowpath for the procedures and techniques performed by the integration module 18. Upon recognition of the custom code 42 (step 100), the integration module 18 retrieves the standard charge rates for the CPT codes that are listed as EHE-related medical billing code(s) in the unique identifier (step 102). The integration module 18 queries the universal chargemaster database 24 and medical billing code database 26 to generate, or otherwise calculate, a universal charge rate for the EHE. As the universal charge rate is generated from the universal chargemaster and from standard CPT codes, the universal charge rate is applicable to all payors.

Next, the integration module 18 attaches, or otherwise associates, the contract rate for a specific payor to the custom code (step 104). The integration module 18 obtains the identity of the payor from (i) the patient historical data, and/or (ii) the payor fee schedule, and/or (iii) from user input. The integration module 18 queries the payor fee schedule database and retrieves the appropriate payor contract rate for the EHE.

The integration module 18 then applies a discount to the universal charge rate (step 106). The integration module 18 calculates the discount as the difference between the universal charge rate and the contract rate. The integration module 18 stores the discount rate in the memory module 14. In an embodiment, the discount is identified, or is otherwise flagged, as a “contractual allowance” in the memory module 14 (step 108).

The calculation of (1) a universal charge rate derived from the universal charge master and/or the standard CPT codes (or other medical billing codes), (2) attachment of the contract rate to the custom code, and (3) the computation and recordation of the discount applied to the universal charge rate ensures that the present system is compliant with medical billing regulations imposed by Medicare. The ability of the present system to make adjustments based on the universal chargemaster and CPT codes and subsequently tie the adjustments to the discount, the discount being recorded and stored in the memory module 14, ensures accurate accounting and compliance with medical billing regulations.

The present system ensures that all payors are always charged the same amount for each individual medical billing code and that any reductions in expected payments are associated with the contracted fee schedule for a specific payor. The present system avoids, or otherwise prevents, billing a discounted rate that is not also offered to Medicare. In this way, the present system reduces, or otherwise eliminates, the risk to a healthcare provider for violating Medicare rules and guidelines which could lead to revocation of the healthcare provider's status as a provider of services to Medicare patients.

The integration module 18 then generates a 1-line entry identifying, or otherwise presenting, the custom code, the procedure name, and the contract rate (step 110, 112).

The billing module 20 applies the 1-line entry to the proper billing format. The billing module then sends the final bill for healthcare services to the interface 18. From the interface 16, the user can send the bill for healthcare services to the payor in either electronic form and/or in hard copy form (printed paper form).

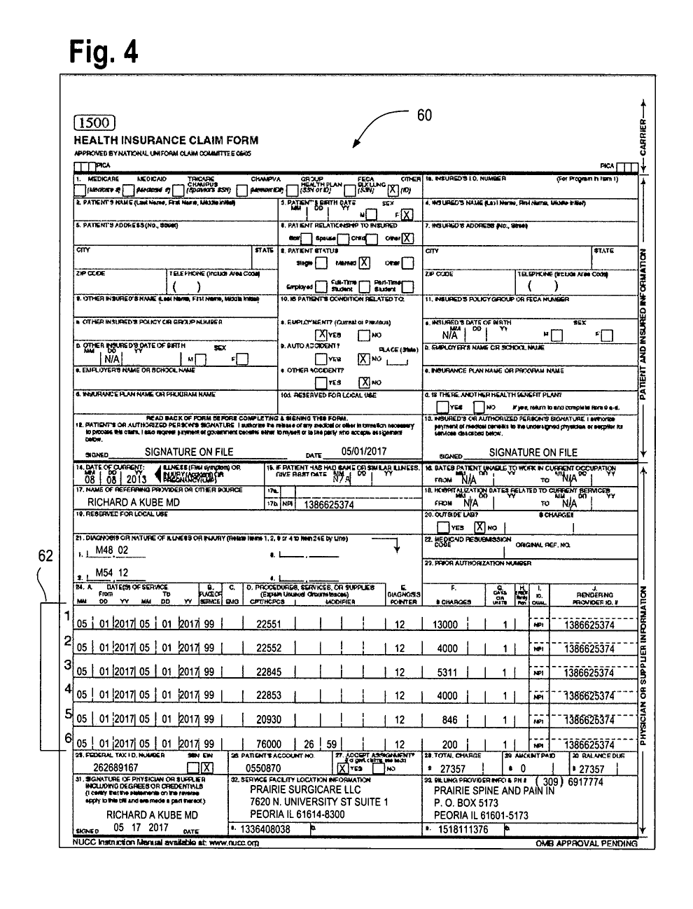

FIG. 4 shows a prior art bill for healthcare services 60. Area 62 of the bill 60 is the description of services section. Area 62 shows a list of six CPT codes. Absent from the bill 60 is a description, in common layman terms, of the medical procedure provided. Bill 60 is unclear and confusing to payors and patients alike. It is understood that bill 60 is only one of the many medical bills received by the patient, with each healthcare provider—the 1HP and one or more 2HPs—sending the patient a respective bill for services/equipment rendered by each healthcare provider related to the administration of the medical procedure.

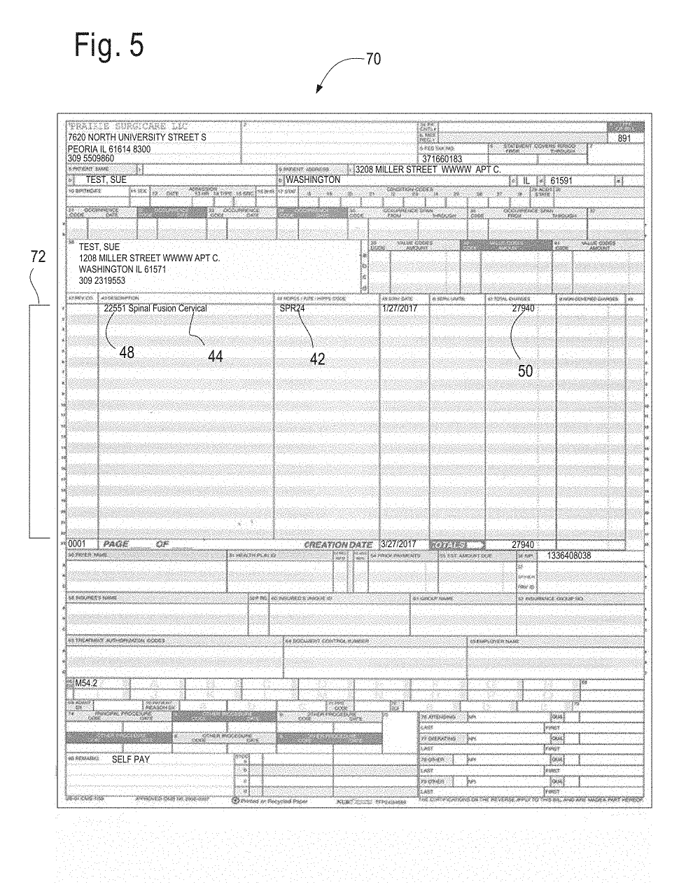

FIG. 5 shows a bill for healthcare services 70 created by the present system 10. Area 72 of the bill 70 is the description of services section. Area 72 provides the custom code 42, procedure name 44, one of the EHE-related CPT codes 48 (for Medicare compliance), and the contract rate 50.

Bill 70 is clear and provides a procedure description that is readily understandable for both patient and payor. Bill 70 provides transparency to the billing of healthcare services. With respect to the EHE, bill 70 is the sole (the only) bill sent to the payor and/or patient.

In an embodiment, the bill 70 is the sole bill sent to the payor and/or to the patient for the EHE, bill 70 delivered to payor and/or patient to the exclusion of any and/or all bills from any 2HP involved in the administration of the EHE. In other words, no 2HPs involved in the EHE send a bill to the payor or patient—the bill generated by the 1HP using the present system 10 is the only bill sent to payor/patient for the EHE.

### 2. Method

The present disclosure provides a method for billing healthcare services. The method includes preparing a list of medical procedures necessary for providing an entire healthcare encounter (EHE) to a patient. The method includes linking the EHE with two or more relevant medical billing codes. The method includes determining a contract rate for the EHE. The method includes creating a unique identifier for the EHE. The unique identifier includes (i) a custom code, (ii) a procedure name, (iii) a description of EHE services, (iv) a list of EHE-related medical billing codes, and (v) the contract rate for the EHE. The method includes calculating a universal charge rate for the EHE. The universal charge rate is based on a universal charge master and the of EHE-related medical billing codes of the unique identifier. The method includes attaching the custom code to the contract rate for a specified payor. The method includes applying a discount for the difference between the universal charge rate and the contract rate. The method includes generating a bill for healthcare services for the specified payor which includes a 1-line entry with the custom code, the procedure name, and the contract rate.

The method includes preparing a list of medical procedures necessary for providing an entire healthcare encounter (EHE) to a patient. In an embodiment, one or more members or employees of the lead healthcare provider (1HP) prepare the EHE. One or more 1HP employees utilize past experience and historical patient data to identify and list all the medical procedures, facilities, and equipment necessary to provide the EHE. The employees of the 1HP also identify all auxiliary healthcare providers, medical subcontractors, medical supply vendors, and healthcare facilities (hospital, operating room, recovery room) necessary to administer the EHE (2HPs). In a further embodiment, the 1HP contracts in advance with each 2HP for the price/cost of each 2HP's contribution to the EHE.

The method includes creating a unique identifier for the EHE. In an embodiment, the 1HP creates a unique identifier for the EHE. The unique identifier includes a custom code, a procedure name, a description of EHE services (EHE descriptor), a list of EHE-related medical billing codes, and a contract rate for each respective EHE. In an embodiment, a user (such as an employee of the 1HP, for example) enters the unique identifier by way of the interface 16 and processing module 12 for storage in the EHE database of the memory module 14.

The method includes linking the EHE with two or more relevant medical billing codes. One or more members of the 1HP utilize historic patient data to determine the medical billing codes that embody procedures of the EHE, or otherwise are relevant to the EHE. The medical billing codes are linked, or are otherwise associated with, or connected to the EHE.

The method includes determining a contract rate for the EHE. One or more members of the 1HP analyze historical patient data and determine the 1HP's cost for performing its portion of the EHE. The 1HP also evaluates the contracts with the 2HPs to determine the overall cost of providing the EHE. The 1HP subsequently calculates a contract rate for the EHE. In an embodiment, the contract rate is approved by one or more payors before the EHE is administered to a patient covered by the payor.

The method includes calculating a universal charge rate for the EHE. The universal charge rate is based on the universal charge master and the EHE-related medical billing codes of the unique identifier. In an embodiment, the 1HP calculates the universal charge rate for the EHE.

The method includes attaching the custom code to the contract rate for a specified payor. In an embodiment, the 1HP attaches the custom code to the contract rate for a specified payor.

The method includes applying a discount for the difference between the universal charge rate and the contract rate. The universal charge rate for the EHE is adjusted, or otherwise is reduced, by an amount to arrive at the contract rate. The difference between the universal charge rate and the contract rate is the discount. In an embodiment, the 1HP computes the discount amount. The 1HP computes the discount amount as the difference between the universal charge rate for the EHE and the contract rate for a specified payor. In a further embodiment, the 1HP records the universal charge rate, the discount, and/or the contract rate for accounting and/or Medicare compliance purposes. Since the contract rate is mutually agreed upon in advance between the 1HP and the payor, and the contract rate, the universal charge rate, and the discount are recorded, the present method meets all compliance standards with all payors including Medicare.

The method includes generating a bill for healthcare services which includes a 1-line entry with the custom code, the procedure name, and the contract rate. It has been found that many payors prefer to see the contract rate on the billed invoice rather than the universal charge rate. The present method allows for the contract rate to appear on the final bill for healthcare services. In an embodiment, the 1HP generates the bill (with the 1-line entry) for healthcare services.

The method includes delivering the bill for healthcare services to the payor and/or patient, the bill having the 1-line entry with the custom code, the procedure name, and the contract rate. In an embodiment, the 1HP delivers the bill for healthcare services to the specified payor and/or patient (electronically and/or paper copy). In a further embodiment, the 1HP delivers the sole (one and only one) bill for the EHE to the payor and/or patient to the exclusion of bills related to the EHE being sent from 2HPs to the payor/patient. In other words, the present method entails the 1HP being the only entity sending a bill for EHE, with no bills from 2HPs being sent to the payor/patient with respect to the EHE.

In an embodiment, method includes recording the universal charge rate for the EHE, recording the discount, and recording contract rate for the EHE.

In an embodiment, one, some, or all of the method steps is/are performed using the system 10. In a further, embodiment, one, some, or all of the method steps is/are performed by the 1HP utilizing the system 10.

The present system and method generates a bill for healthcare service with the following advantageous features:
a 1-line item entry with a custom code, and a short and understandable description of services that is easy to read and understand by a patient and/or a payor; and/or
a single bill for healthcare service with the actual cost of the EHE that is expected to be paid by the payor; and/or
a bill for healthcare services with adjustments and discounts already applied such that the contract rate shown is understood by payor and healthcare provider to be the true and actual cost to be paid by the payor; and/or
a single bill from a single healthcare provider for the EHE, the single medical bill from the 1HP to the payor and to the exclusion of medical bills from any and all 2HPs to the payor.

The present system and method advantageously promote transparency for the billing of healthcare services. The present system and method enable payors and patients to effectively shop for the best healthcare provider price. The present system and method reduce the cost for the delivery of healthcare services while increasing quality and simultaneously remaining CMS (Centers for Medicare and Medicaid Services) compliant.

It is specifically intended that the present disclosure not be limited to the embodiments and illustrations contained herein, but include modified forms of those embodiments including portions of the embodiments and combinations of elements of different embodiments as come within the scope of the following claims.

1. A system for billing healthcare services comprising:
one or more computing devices comprising a memory module, an interface, and a processing module comprising a billing module and an integration module;
the memory module storing
(i) patient historical data;
(ii) a universal charge master database;
(iii) a medical billing code database;
(iv) a payor fee schedule database; and
(v) an entire health encounter (EHE) database, the EHE database comprising
a unique identifier for each EHE, the unique identifier comprising a custom code, a procedure name, a description of EHE services, a list of EHE-related medical billing codes; and a contract rate for the EHE;
wherein receipt of the custom code by the billing module triggers the integration module to
calculate a universal charge rate for the EHE based on the EHE-related medical billing codes and the universal charge master;
attach the custom code to contract rate for a specified payor;
apply a discount for the difference between the universal charge rate and the contract rate; and
generate a bill for healthcare service for the specified payor, the bill comprising a 1-line entry with the custom code, the procedure name, and the contract rate.

2. The system of claim 1 wherein the integration module stores the discount in the memory module.

3. A method for billing healthcare services comprising:
preparing a list of medical procedures necessary for providing an entire healthcare encounter (EHE) to a patient;
linking the EHE with two or more relevant medical billing codes;
determining a contract rate for the EHE;
creating a unique identifier for the EHE, the unique identifier comprising
(i) a custom code,
(ii) a procedure name,
(iii) a description of EHE services,
(iv) a list of EHE-related medical billing codes; and
(v) the contract rate for the EHE;
calculating a universal charge rate for the EHE from a universal charge master and the EHE-related CMS codes;
attaching the custom code to the contract rate for a specified payor;
applying a discount for the difference between the universal charge rate and the contract rate; and
generating a bill for healthcare services for the specified payor, the bill comprising a 1-line entry with the custom code, the procedure name, and the contract rate.

4. The method of claim 3 comprising recording the universal charge, the discount, and contract rate for the FHF.
